(19) United States
(12) Patent Application Publication
(10) Pub. No.: US 2012/0004607 A1
(43) Pub. Date: Jan. 5, 2012

(54) SAFETY SYRINGE

(75) Inventors: Graeme Francis Walton, New South Wales (AU); Allan Walsh, New South Wales (AU)

(73) Assignee: GLOBAL MEDISAFE HOLDINGS PTY LIMITED, The Hill, NSW (AU)

(21) Appl. No.: 11/575,160

(22) PCT Filed: Sep. 13, 2005
(86) PCT No.: PCT/AU05/01385

§ 371 Date: Feb. 6, 2008

(30) Foreign Application Priority Data
Sep. 13, 2004 (AU) 2004905234

(51) Int. Cl. A61M 5/50

(52) U.S. Cl. 604/110

(57) ABSTRACT

A retractable needle safety syringe (11) with plunger locking mechanism is disclosed. Locking means (36) are provided in the body (12) of the syringe to engage the plunger (13) once it is retracted after use, so that the syringe is rendered incapable of being used further. The locking means is preferably provided by a twist lock feature. A region of weakness (24) is also provided in the plunger so that after the needle is retracted into the body of the syringe, and the plunger engaged with the locking means, the plunger is broken off so it cannot be depressed again.

### TECHNICAL FIELD

This invention relates to a safety syringe having a retractable needle for use in the medical or dental profession or in personal drug administration so that the physician, surgeon or other needle operator might be protected from injury by the needle after its use.

### BACKGROUND

The danger of injury and possible infection from the HIV or hepatitis B virus to medical practitioners using needles in the normal course of their business is well documented.

Further, persons who are in the habit of administering drugs to themselves run a severe risk of contacting either of the specified viruses, or indeed contacting other viruses if a needle, once used, is reused in an unsterilised form.

There have been many proposals aimed at reducing the number of so-called needle-stick injuries and various attempts have been made to provide a safe system for disposal of such needles once used, but such prior proposals have had deficiencies.

Various related aspects of this invention are also disclosed in our co-pending application no PCT/AU2004/001496, the whole of which is incorporated herein by reference thereto.

### OBJECT OF THE INVENTION

It is an object of the present invention to provide a syringe which employs a retractable surgical needle, in a sterile manner prior to use, and for permanently storing that surgical needle, once used, in a substantially safe manner. At the very least the invention provides an alternate means for accommodating the needle of a used syringe to protect against accidental injury arising from unwanted contact with the exposed needle once the syringe has been used.

Although the following description generally refers to a syringe of conventional size, no such limitation is intended thereby, and reference to a syringe is meant to encompass any other needle/syringe combination or needle alone including slimline syringes, where, by suitable adaptation, the invention may also be usefully applied.

### DISCLOSURE OF THE INVENTION

According to the present invention there is provided a safety syringe having any retractable needle facility incorporated therein, wherein the needle is caused to retract into the body of the syringe upon completion of injection once it has been used, the syringe comprising a body and plunger, and wherein locking means is provided in the body of the syringe to engage the plunger once it is retracted after use, so that the syringe is rendered incapable of being used further.

Preferably a region of weakness is provided in the plunger, located on the shaft of the plunger in the vicinity of where it extends from the upper end of the syringe body when fully withdrawn, so that after the needle is retracted into the body of the syringe, and the plunger engaged with the locking means, the plunger is broken off so that it cannot be depressed again, thereby further assisting in rendering the syringe incapable of being used again.

Preferably the locking means is provided by a twist lock feature including corresponding threaded regions in the barrel of the syringe and on the plunger, so that after an injection the plunger is withdrawn and locked in position by twisting the plunger to engage the corresponding threaded portions.

Alternatively the locking means is provided by a twist lock feature including corresponding a twist lock bush having helix grooves therein located in the barrel of the syringe and lugs located on the plunger for engagement in the helix grooves, so that after an injection the plunger is withdrawn and locked in position by twisting the plunger to engage the lugs in with the corresponding helix grooves.

Again, as an alternative, the locking means may be provided by a twist lock feature including corresponding a twist lock bush having helix grooves therein located in the barrel of the syringe and lugs located on the plunger for engagement in the helix grooves, so that after an injection the plunger is withdrawn and locked in position by twisting the plunger to cause the lugs to move through the corresponding helix grooves and pass completely though the bush.

Preferably in this embodiment, a permanent lock facility is provided in the form of a ramp or other protrusion to prevent the lugs from returning through the grooves of the bush and hence preventing the plunger from passing back through the bush into the barrel of the syringe.

Although much of the following description refers to a particular safety syringe having a retractable needle with certain features, it will be understood that the twist locking mechanism may be utilised with any syringe having any form of retractable needle facility.

Thus locking means are provided in the body of the syringe to engage the plunger once it is retracted after use, thereby assisting in rendering the syringe incapable of being used again. For example a split locking threaded bush is provided in one embodiment about the inner upper periphery of the body at the end in which the plunger is inserted which mates with a corresponding locking thread located about the periphery of the plunger as described above.

In this way, in combination with a break point provided in the plunger just above the locking thread, the plunger can be locked after retraction and broken off as well to make it impossible to access or use the needle contained in the body of the syringe.

The invention finds application for example in one broad form which provides a syringe having a retractable needle facility incorporated therein, so that in a first storage position prior to use, the needle is safely enclosed within the body of the syringe and in a second in use position, the needle is caused to extend from the body of the syringe so as to function as a useful needle and syringe combination in known fashion, and wherein the needle is then caused to retract again into the body of the syringe, once it has been used, so as to return it to the first storage position; the syringe comprising a body and plunger, wherein the plunger has means associated with the end thereof contained within the body of the syringe to releasably engage a boss of the needle located in the syringe body so as to retain the needle within the syringe body in the first storage position, and wherein the body of the syringe has means located at the end thereof opposite to where the plunger extends from the body for releasably retaining the needle boss in the second in use position so that the needle extends from the syringe body, the plunger being used to bring the needle boss into engagement with the end of the syringe body; the plunger then being able to be disengaged from the needle boss so as to enable the plunger to be withdrawn and thereby take in fluid into the syringe and then to be used to inject same into a patient in known fashion; and when upon completion of injection, and the needle having been withdrawn from the patient, the plunger is once again brought into engagement with the needle boss and withdrawn so as to cause the needle boss to be released from the end of the syringe body so that the needle is once again retracted into the body of the syringe, the needle remaining attached to the end of the plunger.

Preferably the means associated with the end of the plunger to engage the needle boss is provided in the form of a thread, slot or similar arrangement where the engagement and disengagement is accomplished by twisting the plunger relative the needle boss. Alternatively, the means associated with the end of the plunger to engage the needle boss is provided in the form of an interference fit, or complementary ridge/recess facility, so that the engagement/disengagement is accomplished by a push/pull action.

In embodiments where the engagement is by way of a twisting action and a thread is utilised, the thread arrangement may be a conventional thread arrangement or may be a so called luer-lock style thread. It may also a single, double or three start thread, the latter being especially preferred to provide rapid engagement.

Thus, for example in one embodiment, an internal thread may be located in the needle boss, which allows engagement therein of a protrusion such as a tab located about the periphery of a stem designed to extend into the needle boss. Alternatively, the thread may be located on a stem or protrusion extending from the base of the plunger, such as a luer lock type thread, which is able to mate with a ridge about the periphery of the needle boss to achieve engagement therewith.

In another embodiment, the end of the plunger is provided with wire clips or the like, preferably three, which extend downwardly from the end of the plunger and which each have outwardly extending flanges. These are inserted into corresponding slots located in the needle boss so that upon twisting the plunger, the flanges are caused to move into spaces under lips adjacent the slot so as to prevent removal of the plunger from the needle boss except by twisting the plunger back.

Preferably, the thread or slot arrangement is also provided with a locking means in the form of some additional interference fit to facilitate positive control over the needle boss during engagement and disengagement of the needle boss with the end of the syringe body.

Alternatively, in an embodiment where the engagement/disengagement is accomplished by means of a push/pull action, the means to engage the end of the plunger in the needle boss is provided by means of a first ridge located about the periphery of a stem or the like located at the end of the plunger, designed to extend into the needle boss. A complementary recess located about the inner wall of the needle boss allows the first ridge to mate with the recess so that prior to use, the plunger is engaged with the needle boss, so that the boss and hence the needle is held within the syringe body prior to use, and so that the boss and hence needle itself can be moved into position in the end of the syringe body ready for use when the plunger is pushed down.

With advantage, a second larger ridge is preferably located behind the first ridge so that in use the first ridge allows releasable engagement with the needle boss, thereby facilitating relatively easy withdrawal of the plunger from the needle boss after the needle boss is caused to engage in the end of the syringe body ready for use, whereas, after use (ie after the syringe has been used to inject a patient), the plunger is brought once again into engagement with the needle boss, but on this occasion with greater force so as to ensure engagement of the larger second ridge, rather than the smaller first ridge, in the recess, effectively locking the plunger to the needle boss. This not only allows for greater ease of withdrawing the needle boss from its engagement with the end of the syringe, but also provides greater security against the risk that the needle becomes disengaged from the plunger once it is withdrawn and the needle retracted into the syringe body.

It will also be understood that the ridge and recess may be reversed on the plunger and the needle boss to provide in effect mating in reverse geometry to that described above.

Preferably the means to releasably engage the needle boss in the end of the syringe body is provided by a friction fit of sufficient strength to retain the needle boss whilst the needle and syringe is used in known fashion.

Preferably engagement of the needle boss in the end of the syringe is by means of a taper fit. Preferably the needle boss is fluted or has notches and/or ridges to assist in engaging the needle boss especially so as to assist the plunger to engage and disengage therefrom.

Alternatively, a ridge may be provided about the periphery of the needle boss which mates with a corresponding depression about the inner wall of the end of the syringe, to allow releasable engagement of the needle boss therein. Again it will be understood that the relative positions of ridge and recess may be reversed.

Thus in an one preferred embodiment, the means of engagement is provided by a multi-start thread and more preferably by a three start thread having a taper to allow rapid engagement and sufficient locking force.

An additional ridge may be provided in the inner periphery of the syringe body where the needle boss comes into mating contact therewith so as to allow the needle boss to be “locked” temporarily in position, thereby providing security against accidental retraction whilst in use, until such time as the needle boss is once more engaged with the end of the plunger for permanent retraction.

Preferably the end of the syringe body is sealed initially in order to retain sterility, the seal being broken only as the needle is brought down into the in use position by the action of the plunger. Again, with advantage, the seal may be a rubber membrane which effectively reseals once the needle is retracted. This has the advantage that any fluids withdrawn into the body of the syringe also remain therein for disposal without leaking. Alternatively to puncturing the seal, it may be a removable seal in the form of say a cap or other suitable fitting, which itself may be capable of being refitted to retain any fluid contents.

Preferably, a circlip or other protecting sleeve is provided about the exposed periphery of the plunger prior to use, ie in the region where the plunger extends from the body of the syringe in the initial configuration, so as to prevent the plunger being accidentally depressed, rendering the syringe dangerous and breaking any sterile seal before necessary.

Although such a circlip or protecting sleeve could conceivably be utilised once again to prevent the plunger from being depressed after it has withdrawn the needle following use, it is preferred to have instead a region of weakness in the plunger located on the shaft of the plunger in the vicinity of where it extends from the upper end of the syringe body when fully withdrawn, so that after the needle is itself withdrawn into the body, the plunger may be broken off so that it cannot be depressed again. Thus the syringe will be rendered useless and the needle safely contained in the body of the syringe without any means associated therewith to cause it to be extended again from the body of the syringe.

The plunger may be of any suitable structure, although preferably the shaft thereof is reinforced to withstand the twisting forces necessary to engage and disengage the needle boss in the end of the syringe body.

The means for sealing the lower periphery of the plunger, ie so that it acts as a piston in the body of the syringe, may be by any conventional means including a close or interference fit, as well as by means of rubber piston rings or other sealing membrane located about its periphery.

The invention is thus of particular benefit in that the needle is not only safely retracted after use, but is provided in a safe retracted condition prior to use, which can also remain sterile until use. There is no physical contact at all with the needle, the engagement and disengagement thereof in the end of the syringe body being accomplished remotely by the use of the plunger. Coupled with the advantages of resealing the syringe body with replaceable seal or self closing membrane, not only is the risk of needle-stick injuries obviated, but so too are potentially dangerous fluids usefully and safely contained in the body of the syringe.

Snapping off the plunger also renders the syringe both ineffective for subsequent use as well as providing additional safety in that there is no means by which the needle can be caused to re-emerge or extend from the syringe body.

### BRIEF DESCRIPTION OF THE DRAWINGS

The invention may be better understood from the following non-limiting description of preferred embodiments, in which:

FIG. 1a, 1b are a cross sectional views of an embodiment of the invention utilising a three start thread for engagement between the plunger and needle boss, as well as a locking bush to retain the plunger after use;

FIGS. 2a, 2b are a cross sectional views of a further embodiment of the invention utilising three clips and slots for engagement between the plunger and needle boss, as well as a locking bush to retain the plunger after use;

FIG. 3 is a cross-sectional view of a further embodiment employing a push/pull action;

FIG. 4 is a cross-sectional view of an embodiment of the invention showing the needle engaged in the in-use position; and

FIGS. 5a, 5b, 5c, 5d and 5e are cross-sectional views of the components of another embodiment similar in arrangement to that shown in FIG. 4;

FIG. 6 is a detailed cross sectional view of a twist lock bush for use in various embodiments of the invention;

FIG. 7 is a detailed cross sectional view of a plunger for use with the twist lock bush of FIG. 6;

FIG. 8 is cross sectional exploded view of a syringe according to one aspect of the invention, but without the detail of any twist lock feature which may also be added thereto.

### DETAILED DESCRIPTION OF THE DRAWINGS

Referring to the Figures generally, there are shown various syringes generally referenced 11 throughout the Figures, which comprises a syringe body 12, a plunger 13 and a needle 14. The needle 14 is provided with a boss 15 at its upper end.

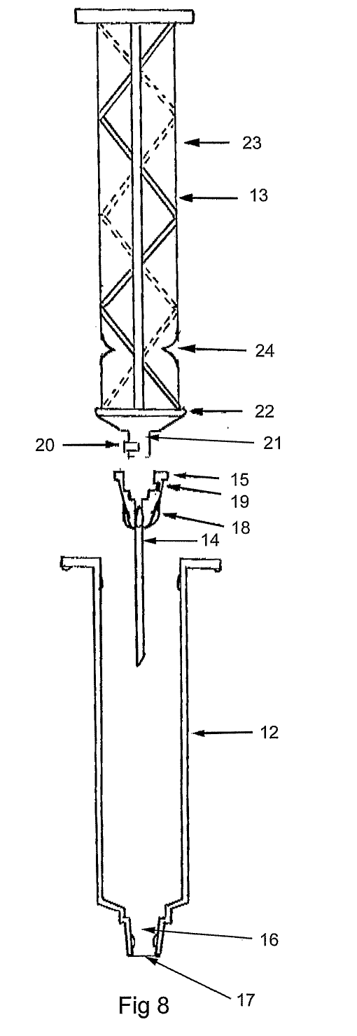

Still referring to the Figures in general, but to FIG. 8 in particular, the syringe body 12 is essentially similar to a conventional syringe body except in so far as the needle 14 is neither permanently connected to the syringe body 12 (as for example in slimline syringes), nor is it attachable to the syringe body 12 from the outside of thereof. Rather, the needle 14 is located initially inside the body of the syringe, as discussed in our co-pending PCT application referenced above. Furthermore, the end region 16 of the syringe body 12 is closed by means of a membrane 17, which may be punctured when the needle 14 is caused to move downwards and into the in use position where the boss 15 thereof occupies the central internal area of tapered end region 16.

The needle 14 is also provided with protrusions or locking web members 18 about boss 15. The locking web members 18, facilitate gripping of the boss 15 in the end region 16 of the syringe body 12, when the needle 14 is forced down into the tapered end region 16.

A locking groove 19 is located about the inner periphery of needle boss 15.

Plunger 13 is provided with a locking tab 20 located on a stem 21 at the base of the plunger 13. The locking tab 20 mates with the locking groove 19, when the stem 21 of the plunger 13 is located in the needle boss 15.

Plunger 13 is also provided with a piston region 22 about its lower periphery which seals against the inside if the syringe body 12, when inserted therein. Although shown here as a fluted arrangement, the plunger 13 may for example be of cylindrical cross section. Such an arrangement would be perhaps more suited to a narrow slimline style syringe. In this embodiment however, the plunger 13 is reinforced with diagonal bracing ridges 23 to provide greater rigidity so as to prevent the plunger 13 itself from twisting as it is turned to engage and disengage the needle boss 15 as described below.

A break groove or weaker region 24 is provided at a suitable location on the plunger 13, so that it may be broken off after it has retracted the needle 14 into the body of the syringe 12, after use as described below.

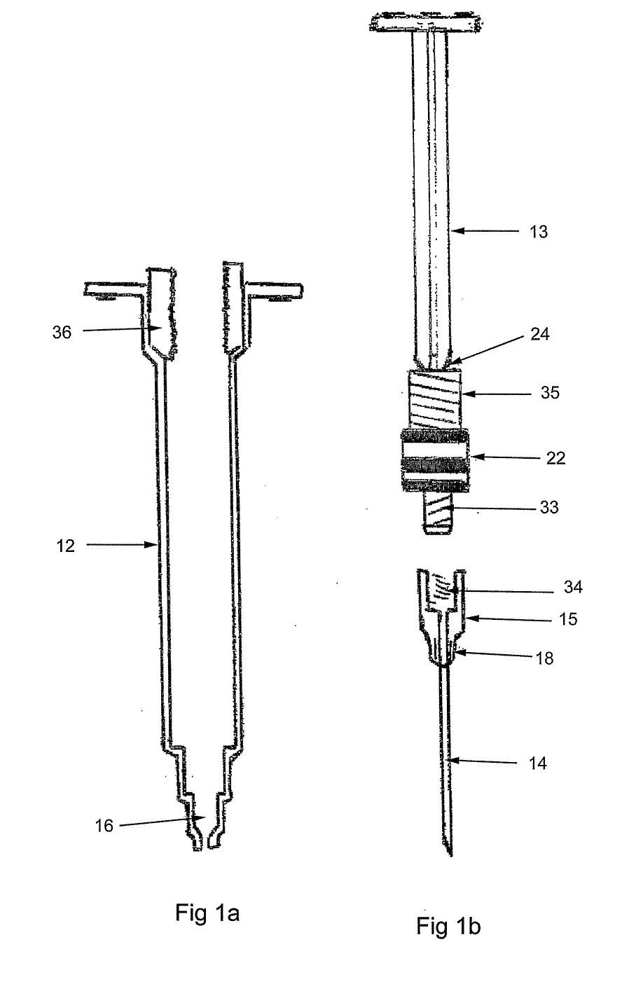

Turning specifically to FIGS. 1a and 1b, there is shown the components which make up a syringe and needle combination which comprises a syringe body 12, a plunger 13 and a needle 14. The needle 14 is provided with a boss 15 at its upper end. The other features common to the all embodiments are referenced with the same reference numerals.

The needle 14 is located initially inside the body of the syringe 12 and is screwed to the plunger 13 by means of the three start (male) thread 33, which mates with an internal (female) threaded region 34 in the needle boss 15. The thread however may be any other suitable single or multi-start thread. It will be understood that in some circumstances it may be preferred to have the male and female threads located so that the male thread is on the needle boss 15, whilst the female thread is located on the end of the plunger 13.

The additional feature of this embodiment is provided by a three start lock thread 35 located in the region between the piston portion 22 and the break of point 24. A corresponding internal lock thread 36 is located in the upper region of the body 12. Again other single multi-start thread arrangements may be utilised.

Thus in use, the plunger 13 is pushed down initially in order to engage the needle 14 (which at this time is engaged with the plunger as mentioned above) into the end region of the barrel 16. The plunger 13 is then retracted so as to draw up the solution to be injected (not shown) into the body 12 of the syringe. After the injection is given, the needle is removed from the patient and the plunger 13 is once again screwed into engagement with the needle 14 so that it may be retracted. Following partial retraction of the plunger 13 and needle 14 into the body 14, the end of the needle 14 may be placed on a hard surface and be caused to bend thereby making it inoperative. The plunger 13 is then drawn fully back and the locking threads 35, 36 are engaged so as to retain the plunger 13 in that position. The plunger is then snapped off at break point 24. With the plunger 13 disabled and the needle 14 bent and refracted inside the body 12 of the syringe it is rendered totally useless and may then be disposed of safely.

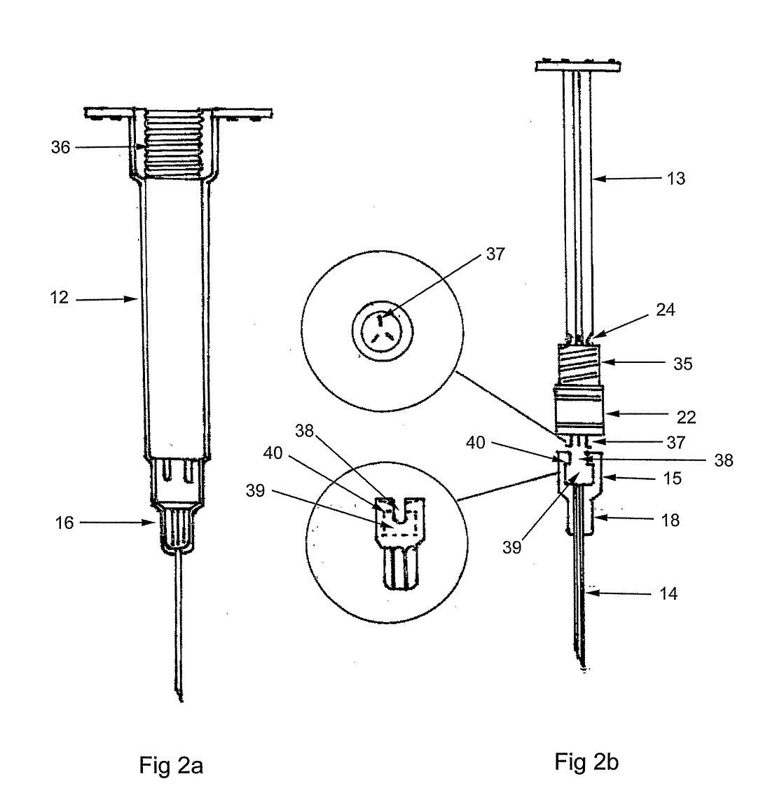

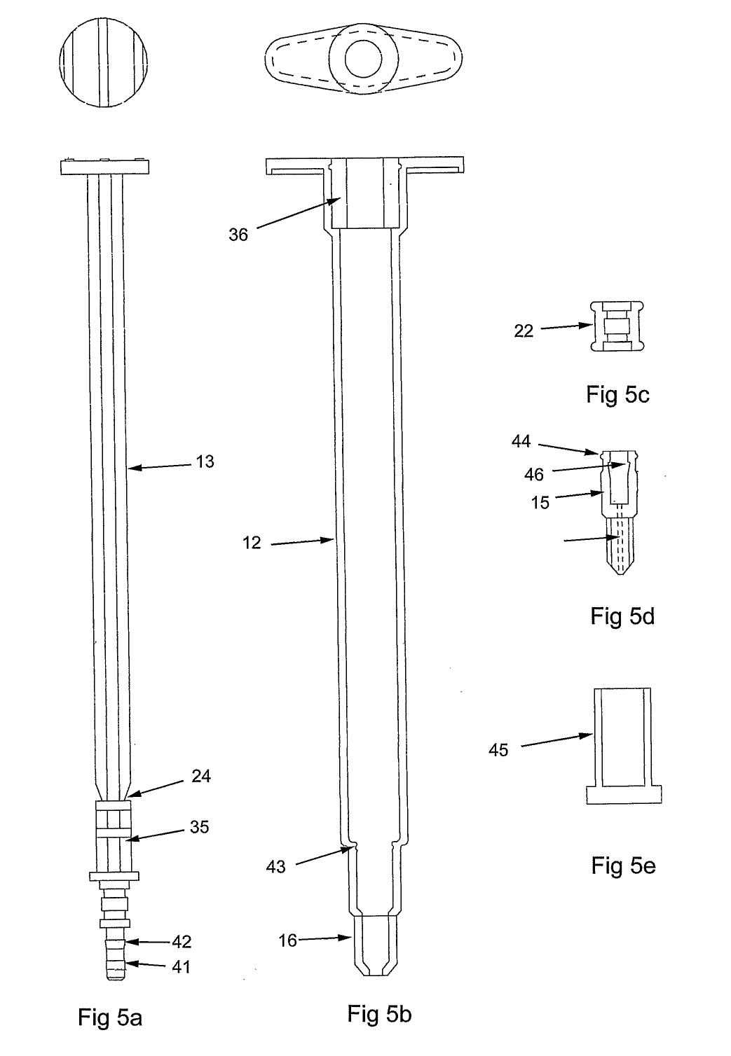

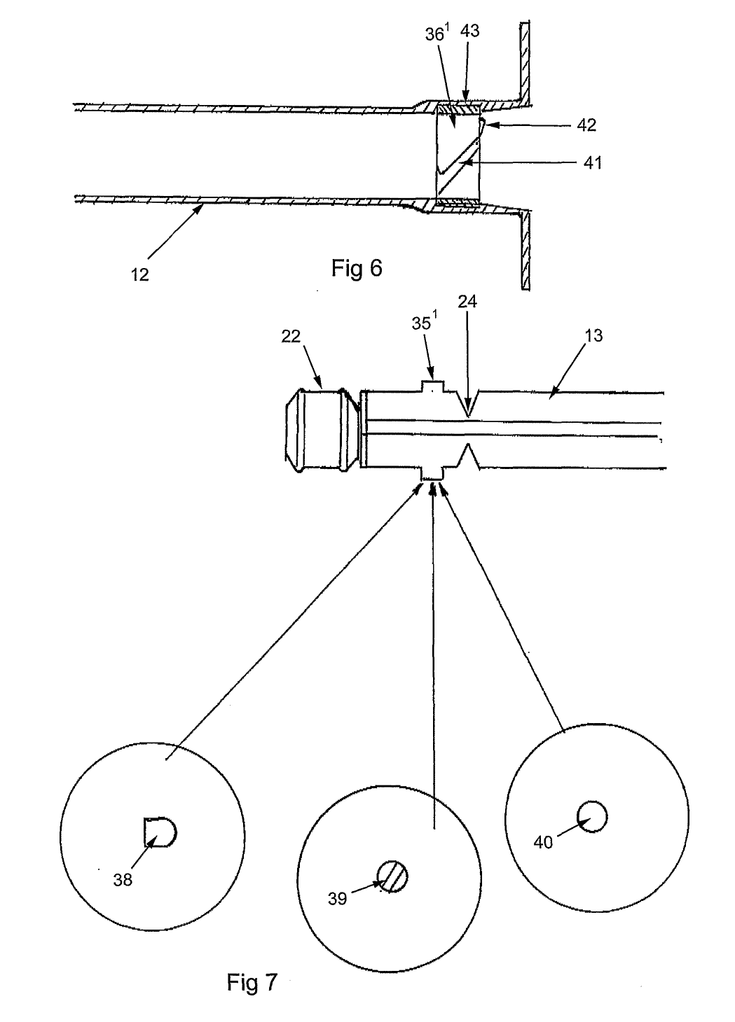

FIGS. 2a and 2b show a further alternate embodiment where like components to those illustrated in the other Figures are again referenced with the same reference numerals. The locking threads 35, 36 shown in the embodiment of FIGS. 5a, 5b are again present in this embodiment. In this case the essential differences again lie in the regions of the needle boss 15 and end of the plunger 13. Specifically the locking tabs and threads of the earlier embodiments are replaced by a wire clips 37 (in this case three) which depend from the end of the plunger 13 as shown. These mate with slots 38 arranged about the top of the needle boss 15 as shown. The slots communicate with a hollow region below 39 so that when the clips 37 are inserted into the slots 38 and the plunger 13 is twisted the ends of the clips 37 are prevented from leaving the needle boss by virtue of the rim or lip 40. Schematically, these are shown in detail in the circled regions of FIG. 6b. Otherwise the basic operation of the embodiment in FIGS. 6a, 6b is similar to that of FIGS. 5a, 5b.

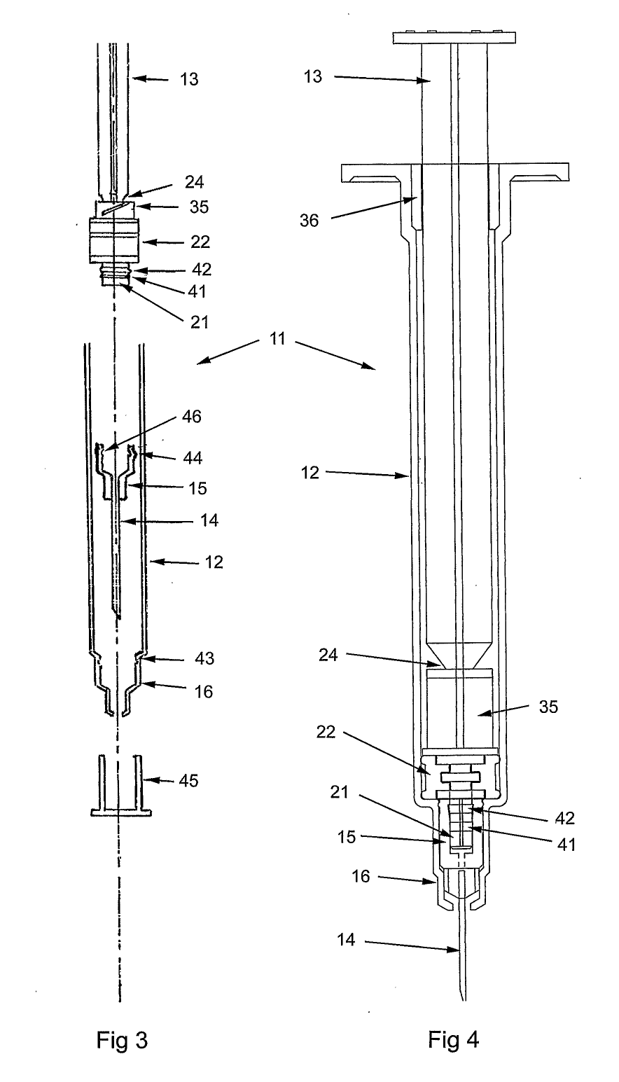

Turning to FIG. 3, there is shown an embodiment of the invention generally referenced 11, comprising a syringe body 12, plunger 13, needle 14 and cap 45. Plunger 13 is provided with a piston region 22 as described above in relation to earlier embodiments. Similarly a break point 24 is located generally above the piston region 22 on the plunger 13. The plunger 13 is further provided with a stem 21, about which are located a first small peripheral ridge 41 and a second larger peripheral ridge 42. A twist locking thread 35 (not specifically illustrated) is also located above the piston region 22, similar to that described in relation to the embodiments of FIG. 6.

The body of the syringe 12 has a recess or depression 43 about the inner wall thereof in the end region 16 of the syringe body 12. Needle 14 has a boss region 15 as described above in relation to earlier embodiments. Located about needle boss 15 is a locking ring or ridge 44 which mates with the aforementioned recess 43 when the boss 15 is brought into engagement with the end 16 of the syringe body 12. Also located about the inner wall of needle boss 15 is an inner groove 46, which receives either of the larger or smaller ridges 41, 42 located on the stem 21 of the plunger 13, when it is brought into engagement with the needle boss 15 as described below.

Prior to use, the needle boss 15 is held temporarily to the plunger 13 by engagement of the small ridge 41 in the inner groove 46 of needle boss 15. When the plunger 13 is pushed down, the needle boss 15 engages with the end 16 of syringe body 12, by means of the engagement of the locking ring 44 in the recess 43. When the plunger 13 is pulled back, the small ridge 41 on the stem 21 of plunger 13 disengages from the inner groove 46 of the needle boss 15, leaving the needle 14 locked in the “out” or in use position. The syringe 11 may then be used in the convention manner.

After use, when the plunger 13 is pushed down hard, the larger ridge 42 engages with the inner groove 46 of the needle boss 15, providing a more permanent lock. When the plunger 13 is then drawn back again , the needle boss 15 comes out of engagement with the end 16 of the syringe body 12, ie locking ring 44 disengages from recess 43, the needle boss 15 and hence needle 14 being held securely onto the plunger 13, buy virtue of engagement of the larger ridge 42 located on the stem 21 of the plunger 13 with inner groove 46 of the needle boss 15.

The plunger 13 is then drawn fully back and by means of the twist lock thread 35 locked as described above in reference to the embodiment of FIG. 6, so that the needle 14 remains securely in the syringe body 12 after use. The plunger is then broken off break point 24 as described in relation to the earlier embodiments

Similar embodiments to that described in principle in FIG. 3 are detailed in FIGS. 4 and 5, where similar features are referenced once again using the same reference numerals where relevant. Referring to FIG. 4, there is shown an assembled syringe 11 of standard dimensions, whilst a similar but slimline version is shown as separate components in FIG. 5. The main points of relevance are the shape of the larger peripheral ridge 41 which in these cases is tapered, which allows for a ratchet like locking when engaged in the correspondingly shaped inner groove 46 of needle boss 15.

Also shown in detail is a sleeve 47 which is located about the plunger 13 to provide the piston region 22 described above. Otherwise, the operation of the embodiments of FIGS. 4 and 5 is essentially the same as that described in relation to the embodiment depicted in FIG. 3.

FIGS. 6 and 7 specifically show in detail a twist lock bush 36¹ and corresponding plunger 13 for use therewith which may be used in any of the aforementioned embodiments, as well as in any safety style retractable syringe 11 where it is desired to render the syringe 11 completely unusable after use by locking the plunger 13 and breaking it off at break point 24 and hence leaving the needle 14 encased in the barrel 12 of the syringe 11 and otherwise inaccessible.

Again features common to previous embodiments are referenced with the same reference numerals.

The plunger 13 is provided with a lugs 35¹ which may be of any suitable cross sectional shape. Three particular sections are shown in detail at 38, 39, 40, being respectively round entrance with flat base, thread segment and simple round.

The bush 36¹ can be split to wrap around the plunger 13. Alternatively it may be solid and screwed over the plunger 13 prior to fitting the piston 22 thereto. The bush 36¹ has two helix internal grooves 41 to feed the lugs 35¹ as the plunger 13 is twisted through the bush 36¹. When the lugs 35¹ come through the bush 36¹ they rise over the permanent lock 42 and the plunger 13 is thereby locked within the barrel 12. With the plunger 13 locked, the piston 22 is displaced jamming it within the barrel 12. As the piston 22 is reduced in depth it expands in diameter. The twist lock bush 36¹ is held in the barrel by groove 43 as shown.

Once it is desired to use the syringe according to any of the embodiments discussed above, the plunger 13 depressed which causes the needle to break the membrane of 17 or to pass through the end of the syringe body, the cap 31 having been removed prior to doing so. The needle boss 15 is caused to locate and lock in the end of the syringe body 12 by twisting the plunger which also disengages the plunger from the needle boss 15. The needle 14 is thus locked in the outer or use position.

The plunger 13 of the syringe 11 is then used to draw up fluid for injection 32 from a reservoir etc not shown in the usual manner.

Once full, the syringe 11 is then used in the convention, where the syringe body 12 is emptied of injectable fluid 32 to inject a patient. After use the needle 14 of the syringe 11 is withdrawn from the patient.

This action once again brings the end of the plunger 13 into contact with the needle boss 15, with which it may be caused to mate once again by twisting the plunger 13 in the opposite direction to that which earlier disengaged it.

The plunger 13 is then be retracted bringing back with it the needle 14 safely into the body of the syringe 12. The plunger 13 is then twisted in the twist lock feature to lock it securely and is broken off at the weaker region 24, thereby rendering it completely useless, and consequently harmless.

It will be appreciated by those skilled in the art that many modifications and variations may be made to the embodiments described herein without departing from the spirit or scope of the invention.

Throughout the specification the word “comprise” and its derivatives are intended to have an inclusive rather than exclusive meaning unless the context requires otherwise.

The invention disclosed herein has utility in that provides an alternate means for a safety syringe having a retractable needle facility which can be rendered useless after an injection by locking the plunger and breaking it off

1. A safety syringe having any retractable needle facility incorporated therein, wherein the needle is caused to retract into the body of the syringe upon completion of injection once it has been used, the syringe comprising a body and plunger, and wherein locking means is provided in the body of the syringe to engage the plunger once it is retracted after use, so that the syringe is rendered incapable of being used further.

2. A safety syringe according to claim 1, wherein a region of weakness is provided in the plunger, located on the shaft of the plunger in the vicinity of where it extends from the upper end of the syringe body when fully withdrawn, so that after the needle is retracted into the body of the syringe, and the plunger engaged with the locking means, the plunger is broken off so that it cannot be depressed again, thereby further assisting in rendering the syringe incapable of being used again.

3. A safety syringe according to either claim 1 or claim 2, wherein the locking means is provided by a twist lock feature including corresponding threaded regions in the barrel of the syringe and on the plunger, so that after an injection the plunger is withdrawn and locked in position by twisting the plunger to engage the corresponding threaded portions.

4. A safety syringe according to either claim 1 or claim 2, wherein the locking means is provided by a twist lock feature including corresponding a twist lock bush having helix grooves therein located in the barrel of the syringe and lugs located on the plunger for engagement in the helix grooves, so that after an injection the plunger is withdrawn and locked in position by twisting the plunger to engage the lugs in with the corresponding helix grooves.

5. A safety syringe according to either claim 1 or claim 2, wherein the locking means is provided by a twist lock feature including corresponding a twist lock bush having helix grooves therein located in the barrel of the syringe and lugs located on the plunger for engagement in the helix grooves, so that after an injection the plunger is withdrawn and locked in position by twisting the plunger to cause the lugs to move through the corresponding helix grooves and pass completely though the bush.

6. A safety syringe according to claim 5 wherein a permanent lock facility in the form of a ramp or other protrusion prevents the lugs from returning through the grooves of the bush and hence preventing the plunger from passing back through the bush into the barrel of the syringe.

7. A safety syringe according to any one of the preceding claims having a retractable needle facility incorporated therein, so that in a first storage position prior to use, the needle is safely enclosed within the body of the syringe and in a second in use position, the needle is caused to extend from the body of the syringe so as to function as a useful needle and syringe combination in known fashion, and wherein the needle is then caused to retract again into the body of the syringe, once it has been used, so as to return it to the first storage position; the syringe comprising a body and plunger, wherein the plunger has means associated with the end thereof contained within the body of the syringe to releasably engage a boss of the needle located in the syringe body so as to retain the needle within the syringe body in the first storage position, and wherein the body of the syringe has means located at the end thereof opposite to where the plunger extends from the body for releasably retaining the needle boss in the second in use position so that the needle extends from the syringe body, the plunger being used to bring the needle boss into engagement with the end of the syringe body; the plunger then being able to be disengaged from the needle boss so as to enable the plunger to be withdrawn and thereby take in fluid into the syringe and then to be used to inject same into a patient in known fashion; and when upon completion of injection, and the needle having been withdrawn from the patient, the plunger is once again brought into engagement with the needle boss and withdrawn so as to cause the needle boss to be released from the end of the syringe body so that the needle is once again retracted into the body of the syringe, the needle remaining attached to the end of the plunger.

8. A safety syringe according to claim 7, wherein the means associated with the end of the plunger to engage the needle boss is provided in the form of a thread, slot or similar arrangement where the engagement and disengagement is accomplished by twisting the plunger relative the needle boss.

9. A safety syringe according to claim 8, wherein the means associated with the end of the plunger to engage the needle boss is provided in the form of a conventional thread arrangement or a luer-lock style thread.

10. A safety syringe according to claim 8, wherein the means associated with the end of the plunger to engage the needle boss is provided in the form of a single, double or three start thread.

11. A safety syringe according to claim 8, wherein the means associated with the end of the plunger to engage the needle boss is provided by wire clips or the like located on the end of the plunger, which extend downwardly from the end of the plunger and which each have outwardly extending flanges, which are able to be inserted into corresponding slots located in the needle boss so that upon twisting the plunger, the flanges are caused to move into spaces under lips adjacent the slot so as to prevent removal of the plunger from the needle boss except by twisting the plunger back.

12. A safety syringe according to any one of claims 8 to 11 wherein the thread or slot arrangement is also provided with a locking means in the form of some additional interference fit to facilitate positive control over the needle boss during engagement and disengagement of the needle boss with the end of the syringe body.

13. A safety syringe according to claim 7, wherein the means associated with the end of the plunger to engage the needle boss is provided in the form of an interference fit, or complementary ridge/recess facility, so that the engagement/disengagement of the plunger with the needle boss is accomplished by a push/pull action.

14. A safety syringe according to claim 13, wherein the engagement/disengagement is accomplished by means of a push/pull action, and the means to engage the end of the plunger in the needle boss is provided by means of a first ridge located about the periphery of a stem or the like located at the end of the plunger, designed to extend into the needle boss, and where a complementary recess located about the inner wall of the needle boss allows the first ridge to mate with the recess so that prior to use, the plunger is engaged with the needle boss, so that the boss and hence the needle is held within the syringe body prior to use, and so that the boss and hence needle itself can be moved into position in the end of the syringe body ready for use when the plunger is pushed down.

15. A safety syringe according to claim 14, wherein a second larger ridge is located behind the first ridge so that in use the first ridge allows releasable engagement with the needle boss, thereby facilitating relatively easy withdrawal of the plunger from the needle boss after the needle boss is caused to engage in the end of the syringe body ready for use, whereas, after use (ie after the syringe has been used to inject a patient), the plunger is brought once again into engagement with the needle boss, but on this occasion with greater force so as to ensure engagement of the larger second ridge, rather than the smaller first ridge, in the recess, effectively locking the plunger to the needle boss.

16. A safety syringe according to either claim 14 or claim 15 wherein the ridge or ridges as the case may be and the recess are reversed with respect to their location on the plunger and the needle boss so as to provide in effect mating in reverse geometry.

17. A safety syringe according to any one of claims 7 to 16, wherein the means to releasably engage the needle boss in the end of the syringe body is provided by a friction fit of sufficient strength to retain the needle boss whilst the needle and syringe is used in known fashion.

18. A safety syringe according to claim 17, wherein the engagement of the needle boss in the end of the syringe is by means of a taper fit.

19. A safety syringe according to either claim 17 or claim 18 in which the needle boss is fluted or has notches and/or ridges to assist in engaging the needle boss so as to assist the plunger to engage and disengage therefrom.

20. A safety syringe according to claim 19, in which a ridge is provided about the periphery of the needle boss which mates with a corresponding depression about the inner wall of the end of the syringe, to allow releasable engagement of the needle boss therein.

21. A safety syringe according to claim 20, in which a ridge is provided about the inner wall of the end of the syringe which mates with a corresponding depression about the periphery of the needle boss, so as to allow releasable engagement of the needle boss with the syringe.

22. A safety syringe according to any one of claims 7 to 21, in which the end of the syringe body is sealed initially in order to retain sterility, the seal being broken only as the needle is brought down into the in use position by the action of the plunger.

23. A safety syringe according to claim 22 in which the seal is a rubber membrane which effectively reseals once the needle is retracted.

24. A safety syringe according to claim 23, in which the seal is a removable seal in the form of a cap or other suitable fitting, which itself is capable of being refitted to retain any fluid contents.

25. A safety syringe according to any one of claims 7 to 24, in which a circlip or other protecting sleeve is provided about the exposed periphery of the plunger prior to use, ie in the region where the plunger extends from the body of the syringe in the initial configuration, so as to prevent the plunger being accidentally depressed, rendering the syringe dangerous and breaking any sterile seal before necessary.

26. A safety syringe according to any one of claims 7 to 25 in which the plunger is reinforced to withstand the twisting forces or push/pull forces as the case may be, necessary to engage and disengage the needle boss in the end of the syringe body.

27. A safety syringe according to any one of claims 7 to 26, wherein the means for sealing the lower periphery of the plunger, so that it acts as a piston in the body of the syringe, is by any conventional means including a close or interference fit, or by means of rubber piston rings or other sealing membrane located about its periphery.

28. A safety syringe substantially as disclosed herein with reference to the drawings.
